(19) United States
(12) Patent Application Publication
(10) Pub. No.: US 2009/0125085 A1
(43) Pub. Date: May 14, 2009

(54) LIGHTING SYSTEM FOR TUBS DESIGNED TO PROVIDE WELL-BEING WITH RELAXATION TECHNIQUES

(75) Inventor: Silvio Salgarelli, Negrar (IT)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: TECHNODESIGN SRL, Negrar (VR) (IT)

(21) Appl. No.: 11/997,148

(22) PCT Filed: Jul. 29, 2005
(86) PCT No.: PCT/IT2005/000453

§ 371 Date: Jul. 28, 2008

(51) Int. Cl. A61H 33/00; A61N 5/06; A61M 21/02

(52) U.S. Cl. 607/90; 600/26; 4/546

(57) ABSTRACT

A lighting system for tubs designed for therapeutic and/or beauty and/or dermatological treatments, or designed to provide well-being with relaxation techniques, comprising rigid tubes made from transparent material, equipped with means for fixing them inside the body of these tubs, the rigid tubes containing a plurality of electronic cards, each comprising lighting elements with variable chromatics.

### TECHNICAL FIELD

This invention concerns a lighting system for tubs designed for therapeutic and/or beauty and/or dermatological treatments or to provide well-being with relaxation techniques.

This lighting system comprises a series of light fittings with variable chromatics which increase the comfort and well-being of the person during the treatment cycle.

The invention in question proposes the use of particular light fittings which can preferably although not necessarily be used together with a transparent wrapping sheet with a high degree of thermal conductivity for beauty and/or well-being treatments designed for body care.

The lighting system according to the invention allows one to make the most of the energy of colours and light for complete psychological-physical well-being, since it uses particular sources of light which develop their chromatic energy in tubs for relaxation and well-being.

The invention in question can be applied in the beauty treatment and well-being sector and in particular in the sector of dry floatation tubs for physical well-being and beauty treatments.

### BACKGROUND ART

The use of tubs for the application of cosmetic and beauty treatments and for relaxation cycles is known.

These tubs, also known as floatation tubs, are provided with an accessory, one of which is the subject of a patent filed in the name of the applicant hereto, consisting of a particular transparent sheet placed above the body containing the liquid, which can be heated by a specific heating unit with temperature control.

For these types of treatment the body of traditional dry flotation tubs is completely covered by this particular waterproof sheet, in close contact with the heated liquid so as to be able to transmit the heat both by conduction and by other convection means to the person lying on the sheet, without however providing any support to the body, and at the same time sealing the water inside the container.

The sheet is made from transparent, flexible, elastic plastic material so as to easily adapt to the shape of a user who lies down on it and is wrapped in it.

These tubs are particularly suitable for dermatological treatments using mud, seaweed, etc., demonstrating considerable flexibility of use in the beauty treatment sector.

Also known to background art in the beauty treatment and well-being centre sector is the use of light sources which can be fitted in traditional tubs for beauty treatments but more commonly the whirlpool type, which provide the tub with a certain chromatic effect.

In some cases, as in the solution described in the American U.S. Pat. No. 2,317,2721, there is just one light source installed in such a way that a single light projector directs its light beam inside the tub.

To use this solution it is necessary to make a hole in the body of the tub before fitting the projector and then seal the hole to prevent water leakage.

The limitation of this solution concerns the fact that the light source is punctiform, and the luminous effect cannot therefore be diffused, with a very limited chromatic effect and light distribution.

According to other solutions, such as for example the one proposed in the European patent EP01093783, the light source consists of a plurality of lights distributed over the entire inner surface of the tub. This case, however, presents certain installation problems, since a series of holes have to be made in the body of the tub, each being designed to accommodate a single light source, and in particular water seal problems since although each hole in the tub is sealed and siliconed they represent a potential danger from the point of view of infiltrations and water leakage, which can occur above all with the passage of time or as a result of deterioration caused by the water.

### DESCRIPTION OF THE INVENTION

This invention concerns a tub for personal treatments designed to provide well-being with relaxation techniques that can eliminate or significantly reduce the problems described above relative to the light sources commonly used in tubs, employing a new type of lighting system.

This invention proposes in particular to provide a new type of lighting system that can be used in any type of tub for physical well-being and beauty treatments.

This is achieved by means of a lighting system that can be fitted in bath tubs or in particular tubs for beauty treatments and/or for carrying out personal well-being techniques, with the features described in the main claim.

The dependent claims describe advantageous embodiments of the invention.

The lighting system according to the invention comprises rigid tubular pipes in transparent material, equipped with means for fixing them inside the body of a tub for physical well-being and beauty treatments; these rigid pipes, being in direct communication with the outside of the tub, can be fitted internally with a plurality of electronic cards each comprising lighting elements with variable chromatics.

According to the invention these electronic cards, consisting of printed circuits provided with lighting elements formed by LEDs, can be positioned adjacent to each other inside the transparent tubes, using rapid-connection terminal boards which make them easy and quick to change when, for example, the elements are faulty or damaged.

For example, each printed circuit can comprise twelve groups of three different colour LEDs, indicatively red, green and blue.

The luminous tubular elements remain immersed in the water in the tub and, according to a particularly advantageous embodiment of the invention, can be used in conjunction with transparent flotation sheets for tubs used for beauty treatments or relaxation techniques.

When the user lies down in the tub, his/her body is wrapped in the sheet in a floating condition without coming into contact with the water, and this situation activates the lighting system in question which makes it possible to complete the treatment, since the diffused variable chromatic lighting, gives the user a luminous perception with beneficial effects during the treatment, also from the psychological-emotive point of view.

### DESCRIPTION OF THE DRAWINGS

Other features and advantages of the invention will become evident on reading the following description of one embodiment of the invention, given as a non-binding example, with the help of the accompanying drawings in which:

FIG. 1 shows a transparency side view of a tub equipped with lighting means contained inside transparent tubular elements;

FIG. 2 is a schematic view of one of the modular lighting elements which can be joined together;

FIG. 3 represents a schematic view of a detail of one of the ends of the transparent tubes containing a lighting card;

FIG. 4 shows the diagram of a modular card fitted with variable chromatic LEDs and the relative microprocessor control system.

### DESCRIPTION OF ONE EMBODIMENT

In the figures, the reference number 10 indicates in general a tub for beauty, dermatological or other types of treatments, the tub preferably being provided with a wrapping sheet with a high degree of heat conduction, in the case in question a sheet fixed to the body of the tub for physical well-being and beauty treatment.

The main feature of the invention is the presence of a lighting system which is designed to generate diffused lighting with variable chromatics inside the tub to give the user psychological-emotional benefits during the treatment.

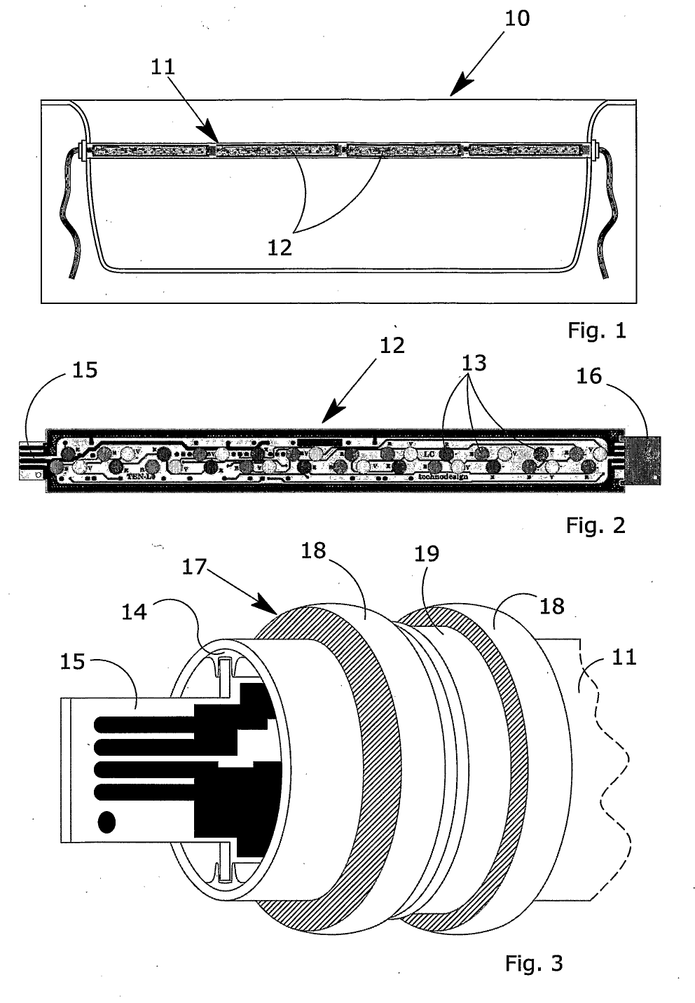

As can be seen in FIGS. 1 and 3 the lighting system uses particular components, the main elements being:

One or more transparent tubular elements 11 made from polycarbonate, fibre glass reinforced plastic, glass, Plexiglas or other appropriate materials, which contain a plurality of electronic cards 12 each equipped with several luminous elements or LEDs, indicated with 13 in FIG. 2, with variable chromatics.

This system makes it possible to obtain lighting whose main feature is that it is distributed in a perfectly uniform way while ensuring maximum waterproofing of the system in terms of water leakage.

The electronic cards are in fact inserted inside tubular elements 11 by means of appropriately shaped tracks 14 which hold the cards in place and allow them to be inserted one after the other.

Quick-coupling means are used to connect the cards to each other, and can be the bayonet type or simple male-female couplings. The male-female couplings 15 and 16 are positioned at the ends of the cards, so that it is sufficient to press them together to achieve the electrical connection of the couplings.

The transparent tubular elements 11 are fixed permanently inside the tub. Holes are made in the walls of the tub (2 holes for each tube containing hundreds of LEDs) which allow the tubes to be inserted horizontally.

The ends of the tubular elements protrude on the outside of the tub and to ensure their fixing and sealing against any water leakage special seals 17 are used, consisting of substantially annular elements with raised edges 18 and a central groove 19.

When the tubular element is fitted, the hole made in the wall of the tub surrounds the central groove 19, while the transparent tubular element 11 remains inserted in the seal 17, forming a perfectly waterproof junction.

The ends of the tubular elements 11 are fitted with particular electrical couplings to which cables are attached that are connected to an electronic control unit or to a PLC or other appropriate control unit.

As already mentioned, each card can consist of a printed circuit comprising groups of LEDs. For example, there can be twelve groups of three different coloured LEDs, indicatively red, green and blue.

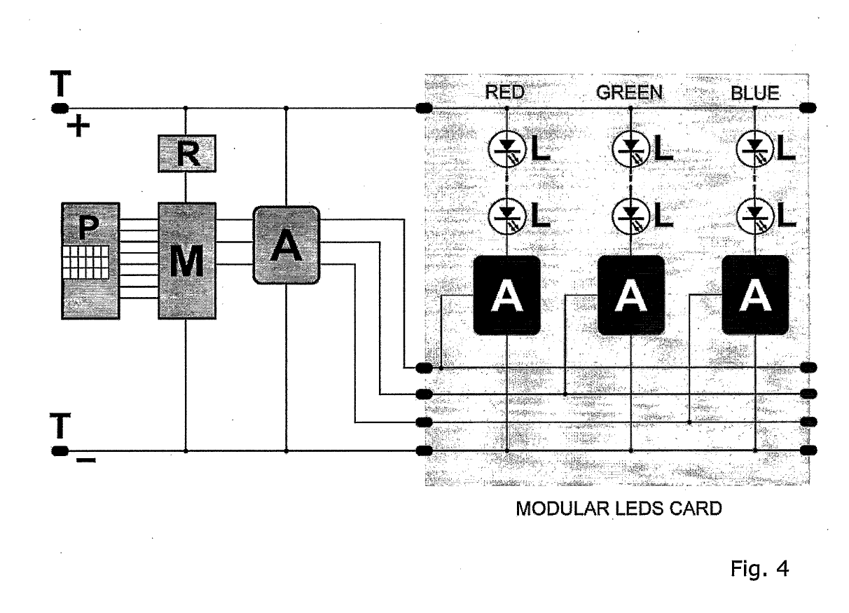

FIG. 4 shows the wiring diagram of a modular element that makes up each group of three LEDs which, in the case in question, are red, green and blue.

For the lighting system to function, it is sufficient to programme the control unit to modulate the luminous intensity and the different shades according to a practically infinite number of combinations, with notable benefits for the user.

If the light sources are combined with the transparent flotation sheet, when a user lies on the sheet its base is deformed, wrapping itself around the user and thus ensuring good adhesion to the user's skin, while the light sources emit their colours according to the programmed intensities.

This condition favours heat exchange with the warm liquid contained in the tub and the simultaneous light emissions make it possible, thanks to the transparency of the sheet, to achieve psychological-perceptive benefits with all the consequent advantages for the result of the treatment session.

Advantageously, the luminous elements can also be used in sensory deprivation tubs or in tubs in which treatment is preferably carried out with the person motionless.

The invention is described above with reference to a preferred embodiment.

It is nevertheless clear that the invention is susceptible to numerous variations within the framework of technical equivalents.

1: A lighting system for a tub for at least one of therapeutic, beauty or dermatological treatment, well-being, or relaxation comprising rigid tubes made from transparent material, fixable inside a body of a tub, the rigid tubes having a plurality of electronic cards, each card including lighting elements with variable chromatics.

2: A lighting system as claimed in claim 1, wherein the electronic cards include a printed circuit whose lighting elements include LEDs of different colors.

3: A lighting system as claimed in claim 1, wherein the electronic cards are positioned alongside each other inside the transparent tubes, including a rapid connection terminal board to facilitate interchange.

4: A lighting system as claimed in claim 1, wherein the electronic cards are inserted in the tubes by tracks for holding the cards in place and allowing them to be inserted one after the other.

5: A lighting system as claimed in claim 1, wherein the electronic cards are connected together with at least one of bayonet type coupling means or male-female couplings positioned at the ends of the cards wherein one card is pushed against the next to achieve the electrical connection of the couplings.

6: A lighting system as claimed in claim 1, wherein the ends of the tubes protrude outside the tub, seals affix and seal against water leakage, the seals including substantially annular elements with raised edges and a central groove.

7: A lighting system as claimed in claim 1, wherein the ends of the tubes include an electrical coupling for cable attachment to an electronic control unit or to a PLC.

8: A lighting system for a tub for at least one of therapeutic, beauty or dermatological treatment, well-being, or relaxation comprising a rigid tube having a transparent material fixable inside a body of a tub, the rigid tube having an electronic card, the card including a lighting element with variable chromatics.

9: A lighting system as claimed in claim 8, wherein the electronic cards include a printed circuit whose lighting elements include LEDs of different colors.

10: A lighting system as claimed in claim 8, wherein the electronic cards are positioned alongside each other inside the transparent tubes, including a rapid connection terminal board to facilitate interchange.

11: A lighting system as claimed in claim 8, wherein the electronic cards are inserted in the tubes by tracks for holding the cards in place and allowing them to be inserted one after the other.

12: A lighting system as claimed in claim 8, wherein the electronic cards are connected together with at least one of bayonet type coupling means or male-female couplings positioned at the ends of the cards wherein one card is pushed against the next to achieve the electrical connection of the couplings.

13: A lighting system as claimed in claim 8, wherein the ends of the tubes protrude outside the tub, seals affix and seal against water leakage, the seals including substantially annular elements with raised edges and a central groove.

14: A lighting system as claimed in claim 8, wherein the ends of the tubes include an electrical coupling for cable attachment to an electronic control unit or to a PLC.
